(19) United States
(12) Patent Application Publication
(10) Pub. No.: US 2020/0147147 A1
(43) Pub. Date: May 14, 2020

(54) MICRON FISH OIL COMPOSITION, AND PREPARATION PROCESS AND USES THEREOF

(71) Applicant: TCI CO., LTD, Taipei City (TW)

(72) Inventors: Yung-Hsiang LIN, Taipei City (TW); Chen-I KAO, Taipei City (TW)

(21) Appl. No.: 16/611,663

(22) PCT Filed: May 23, 2018
(86) PCT No.: PCT/CN2018/088044

§ 371 Date: Nov. 7, 2019

Related U.S. Application Data
(60) Provisional application No. 62/510,214, filed on May 23, 2017.

(51) Int. Cl. A61K 35/60; A61K 47/36; A61K 9/107

(52) U.S. Cl. CPC A61K 35/60; A61K 9/1075; A61K 47/36

(57) ABSTRACT

The present disclosure provides a micron fish oil composition and a method for preparing the micron fish oil composition. The method includes mixing a composite plant colloid with a fish oil and subjecting to an emulsification treatment to obtain an emulsion; and homogenizing the emulsion at a pressure of 300 bar to 350 bar to obtain the micron fish oil composition. The present disclosure also provides a method for enhancing bioabsorbability and bioavailability in a subject, including administering to the subject in need thereof a food product including an effective amount of the micron fish oil composition.

### CROSS-REFERENCE TO RELATED APPLICATION

This application claims priority of U.S. patent application No. 62/510,214, filed on May 23, 2017, the content of which is incorporated herein in its entirety by reference.

### BACKGROUND OF THE INVENTION

### 1. Field of the Invention

The present invention relates to a micron fish oil composition and a method for preparing the micron fish oil composition, particularly to a method for enhancing a bioabsorbability and a bioavailability in a subject, comprising administering to the subject in need thereof a food product comprising an effective amount of the micron fish oil composition.

### 2. The Prior Art

Fish oil is a fat that is taken from fat-rich fish. For example, salmon, mackerel, herring or sardine are oil-rich fish. Fish oil contains omega-3 fatty acids, which are mainly two kinds of unsaturated fatty acids: eicosa pentanoic acid (EPA) and docosahexaenoic acid (DHA). Eicosa pentanoic acid and docosahexaenoic acid are important fatty acids that form phospholipids and cholesterol esters, having many effects, such as lowering high cholesterol, lowering breast cancer risk, improving Alzheimer's disease and Parkinson's disease, even can help to improve mental illnesses such as depression and anxiety.

In order to supplement EPA and DHA, many people would take supplements containing fish oil directly. At present, most of the food products containing fish oil on the market are in the form of capsules, and the people who supplement the fish oil are mostly the elderly and children. The food products in the form of capsules are prone to being difficult to swallow and increase the risk of picking up. In addition, food products that generally contain fish oil are not good for human body absorption and availability, and do not take into account the absorption rate of omega-3 fatty acids, which causes the concentration of EPA and DHA taken into the human body to be inconsistent with health care needs. Therefore, if a micron fish oil composition that can enhance the bioabsorbability and bioavailability in a subject can be developed, it will bring great blessings to users who have such a need and further breakthroughs in the art.

### SUMMARY OF THE INVENTION

A primary objective of the present invention is to provide a method for preparing a micron fish oil composition, comprising the following steps: (a) mixing a composite plant colloid with a fish oil and subjecting to an emulsification treatment to obtain an emulsion; and (b) homogenizing the emulsion at a pressure of 300 bar to 350 bar to obtain the micron fish oil composition.

Another objective of the present invention is to provide a micron fish oil composition prepared by a process comprising the steps of: (a) mixing a composite plant colloid with a fish oil and subjecting to an emulsification treatment to obtain an emulsion; and (b) homogenizing the emulsion at a pressure of 300 bar to 350 bar to obtain the micron fish oil composition.

Another objective of the present invention is to provide a method for enhancing a bioabsorbability and a bioavailability in a subject, comprising administering to the subject in need thereof a food product comprising an effective amount of the aforesaid micron fish oil composition.

According to an embodiment of the present invention, the composite plant colloid is selected from the group consisting of arabic gum, guar gum, xanthan gum, and any combination thereof.

According to an embodiment of the present invention, the composite plant colloid is in an amount of 6% to 10% by weight, and the fish oil is in an amount of 3% to 15% by weight. Preferably, the arabic gum is in an amount of 5.6% to 8.4% by weight; the xanthan gum is in an amount of 0.16% to 0.24% by weight; the guar gum is in an amount of 0.064% to 0.096% by weight; the fish oil is in an amount of 7.2% to 10.8% by weight.

According to an embodiment of the present invention, the micron fish oil composition has an average particle size between 180 and 250 nm.

According to an embodiment of the present invention, the emulsification treatment has a time duration between 25 and 45 minutes. Preferably, the time duration of the emulsification treatment is 35 minutes.

According to an embodiment of the present invention, the emulsification treatment is carried out at 15° C. to 30° C. Preferably, the emulsification treatment is carried out at 25° C.

According to an embodiment of the present invention, the homogenizing step is carried out at 15° C. to 40° C. Preferably, the homogenizing step is carried out at 25° C. to 30° C.

In summary, The micron fish oil composition of the present invention has the effect of good emulsion stability compared with other branded emulsified fish oils, is not easy to produce oil-water separation, can enhance bioabsorbability and bioavailability in a subject, has an average particle size lower than the average particle size of the known fish oil products, thereby increasing the absorption rate and utilization rate in the subject for EPA and DHA.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 is a chromatogram showing the micron fish oil composition of the present invention (i.e., experimental group).

FIG. 2 is a chromatogram showing the fish oil product (i.e., comparative group) purchased from Melaleuca Co., Ltd.

FIG. 3 is a photograph showing the appearance of the micron fish oil composition of the present invention by the emulsification treatment.

FIG. 4 is a photograph showing the appearance of the fish oil product purchased from Melaleuca Co., Ltd. by the emulsification treatment.

FIG. 5 shows the result of TSI analysis of the emulsification treatment of the micron fish oil composition of the present invention (i.e., experimental group) and the fish oil product (i.e., comparative group) purchased from Melaleuca Co., Ltd.

FIG. 6 shows the result of particle size analysis of the micron fish oil composition of the present invention after homogenizing (i.e., experimental group).

FIG. 7 shows the result of particle size analysis of the fish oil composition without homogenizing (i.e., control group).

FIG. 8 shows the result of particle size distribution of the micron fish oil composition of the present invention after homogenizing (i.e., experimental group).

FIG. 9 shows the result of particle size distribution of the fish oil composition without homogenizing (i.e., control group).

FIG. 10 shows changes in plasma EPA concentration in the subject after administration of the micron fish oil composition (i.e., experimental group) of the present invention by high pressure (300 bar to 350 bar) homogenization or the fish oil composition (i.e., control group) which has not been subjected to high pressure homogenization, in which “#” indicates p<0.1, and “*” indicates p<0.05.

FIG. 11 shows changes in plasma DHA concentration in the subject after administration of the micron fish oil composition (i.e., experimental group) of the present invention by high pressure (300 bar to 350 bar) homogenization or the fish oil composition (i.e., control group) which has not been subjected to high pressure homogenization, in which “#” indicates p<0.1, and “*” indicates p<0.05.

FIG. 12 shows changes in EPA concentration in red blood cells (RBC) in the subject after administration of the micron fish oil composition (i.e., experimental group) of the present invention by high pressure (300 bar to 350 bar) homogenization or the fish oil composition (i.e., control group) which has not been subjected to high pressure homogenization, in which “#” indicates p<0.1, and “*” indicates p<0.05.

FIG. 13 shows changes in DHA concentration in red blood cells in the subject after administration of the micron fish oil composition (i.e., experimental group) of the present invention by high pressure (300 bar to 350 bar) homogenization or the fish oil composition (i.e., control group) which has not been subjected to high pressure homogenization, in which “#” indicates p<0.1, and “*” indicates p<0.05.

FIG. 14 shows changes in plasma EPA and DHA concentration in the subject after administration of the micron fish oil composition (i.e., experimental group) of the present invention by high pressure (300 bar to 350 bar) homogenization or the fish oil composition (i.e., control group) which has not been subjected to high pressure homogenization, in which “#” indicates p<0.1.

FIG. 15 shows changes in EPA and DHA concentration in red blood cells (RBC) in the subject after administration of the micron fish oil composition (i.e., experimental group) of the present invention by high pressure (300 bar to 350 bar) homogenization or the fish oil composition (i.e., control group) which has not been subjected to high pressure homogenization, in which “#” indicates p<0.1.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

The food product according to the present invention can be prepared as a dispersible powder or a granule, a solution, a suspension, an emulsion, and the like.

According to the present invention, the types of the food product include, but are not limited to, health foods and nutritional supplements.

According to the present invention, the term “bioabsorbability” means a capacity to be taken in and made part of an existent whole, to be assimilated, or to be dissolved by an organism.

According to the present invention, the term “bioavailability” refers to the rate and extent to which the active ingredient or active moiety is absorbed from a food product and becomes systematically available.

### Example 1

### Preparation of Micron Fish Oil Composition

First, 6% to 10% by weight of the composite plant colloid (including arabic gum, xanthan gum and guar gum, wherein the content of arabic gum is 7% by weight, the content of xanthan gum is 0.2% by weight, and the content of guar gum is 0.08% by weight) was prepared, boiled in boiling water, and then mixed and stirred at 3,000 rpm for 2 hours to obtain a mother liquor of the composite plant colloid. The mother liquor of the composite plant colloid was cooled to room temperature (25° C.), 10% by weight to 15% by weight of glycerol was added, and 3% by weight to 15% by weight of fish oil (preferably 9% by weight) was added, followed by mixing and stirring at 3,000 rpm for emulsification treatment for 25 minutes to 45 minutes to obtain an emulsion. Thereafter, the emulsion was homogenized at a pressure of 300 bar to 350 bar to obtain the micron fish oil composition of the present invention.

### Example 2

### Analysis of Stratification and Stability of Micron Fish Oil Composition

This experiment was commissioned to the Food Industry Research and Development Institute of Taiwan. Among them, the micron fish oil composition of the present invention was used as an experimental group, and the fish oil product purchased from Melaleuca Co., Ltd. was used as a comparative group.

### 2.1 Chromatographic Analysis

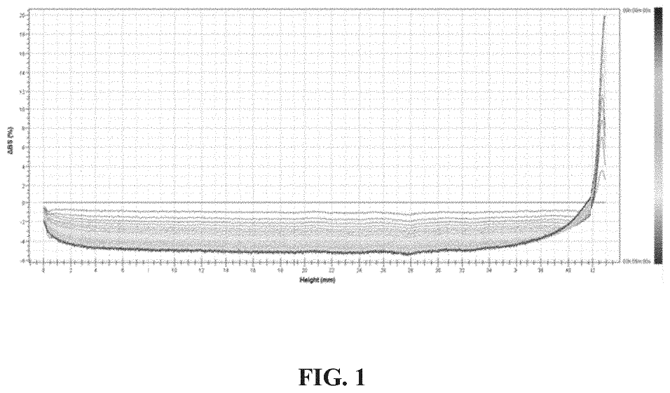

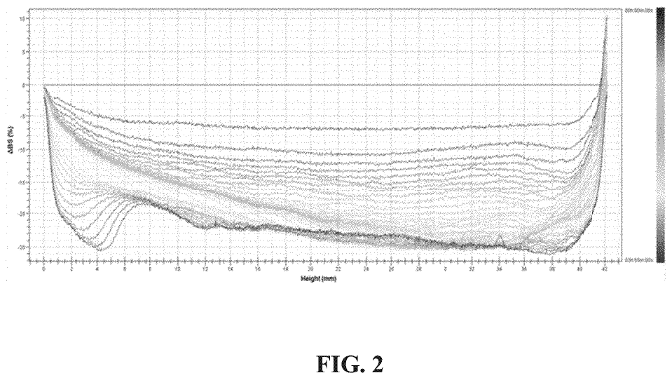

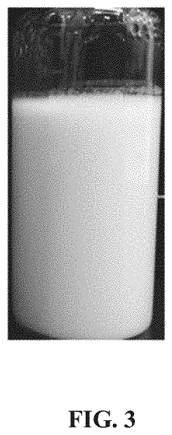

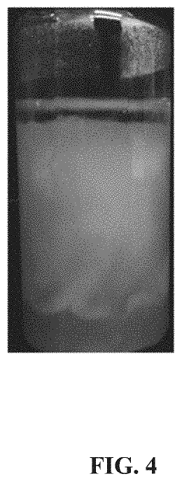

First, chromatographic analysis was carried out at 55° C. and 4 hours using a Turbiscan dispersion stability analyzer. The value of the chromatographic analysis is proportional to the degree of light transmission of the emulsified sample. The results of this experiment are shown in FIG. 1 to FIG. 4. FIG. 1 is a chromatogram showing the micron fish oil composition of the present invention (i.e., experimental group); FIG. 2 is a chromatogram showing the fish oil product (i.e., comparative group) purchased from Melaleuca Co., Ltd.; FIG. 3 is a photograph showing the appearance of the micron fish oil composition of the present invention by the emulsification treatment; and FIG. 4 is a photograph showing the appearance of the fish oil product purchased from Melaleuca Co., Ltd. by the emulsification treatment. As shown in FIG. 1 and FIG. 2, the chromatographic distribution of the experimental group is more concentrated than that of the comparative group, and the chromatographic distribution of the control group is more severe. As shown in FIG. 3 and FIG. 4, compared with the comparative group, the stratification of the experimental group after emulsification treatment is not obvious, and the comparative group has obvious stratification after emulsification treatment. The results of this experiment show that the emulsified condition of the micron fish oil composition of the present invention is quite stable.

### 2.2 Determination of Turbiscan Stability Index (TSI)

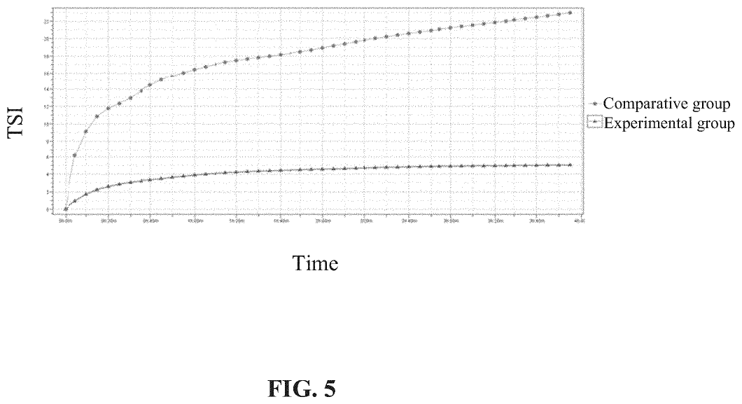

Similarly, the analysis was carried out at 55° C. and 4 hours using the Turbiscan dispersion stability analyzer, and TSI values (stabilized kinetic parameters) were integrated. The TSI value reflects the stability of the emulsified sample. When the TSI value is higher, representing the stability variation is higher, and the dispersion of the emulsified sample is more unstable. FIG. 5 shows the result of TSI analysis of the emulsification treatment of the micron fish oil composition of the present invention (i.e., experimental group) and the fish oil product (i.e., comparative group) purchased from Melaleuca Co., Ltd. As shown in FIG. 5, the TSI values of the comparative group are increasing during the test at 55° C. and 4 hours, which means that the dispersion stability of the comparative group is poor; on the contrary, the curve of the experimental group tends to be stable after 2 hours, which means that the stability variation of the experimental group is less during the test. The results of this experiment show that the micron fish oil composition of the present invention has better dispersion stability. Therefore, the chromatographic analysis and the TSI (Turbiscan stability index) analysis show that the micron fish oil composition of the present invention has good emulsion stability and is less prone to oil-water separation than the comparative group (commercially-branded emulsified fish oil).

### Example 3

### Particle Size Analysis of Micron Fish Oil Composition

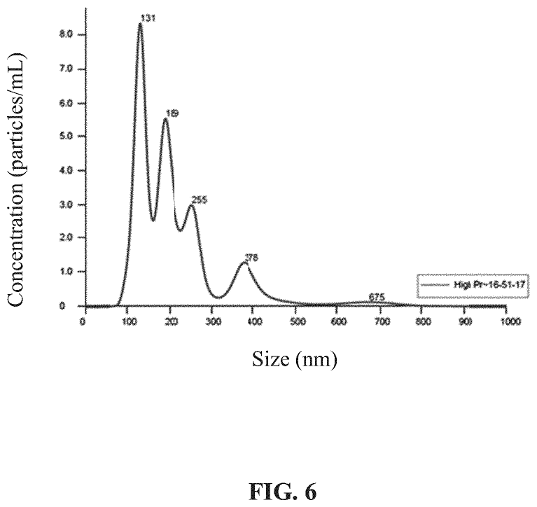

The micron fish oil composition was homogenized using a high pressure homogenizer (GEA Niro Soavi) at a pressure of 300 bar to 350 bar as an experimental group. A fish oil composition which was not subjected to homogenization treatment was used as a control group. Next, the particle size of the experimental group and the control group was examined by DKSH Taiwan Ltd., and the NanoSight NS300 analyzer was used for detection. The NanoSight NS300 analyzer can quickly and automatically analyze the particle size distribution and concentration of all types of nanoparticles from 10 nm to 2000 nm in diameter. The results of this experiment are shown in FIG. 6 to FIG. 9.

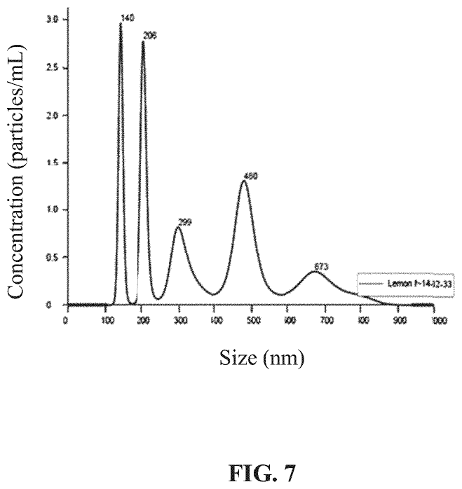

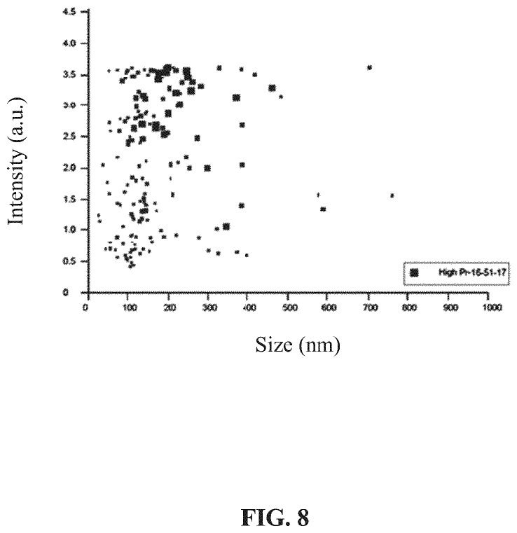

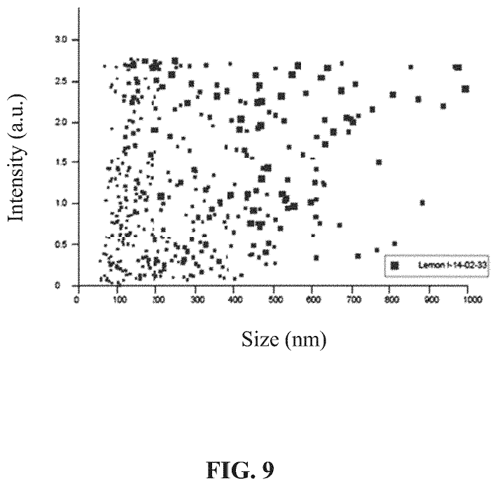

FIG. 6 shows the result of particle size analysis of the micron fish oil composition of the present invention after homogenizing (i.e., experimental group); FIG. 7 shows the result of particle size analysis of the fish oil composition without homogenizing (i.e., control group); FIG. 8 shows the result of particle size distribution of the micron fish oil composition of the present invention after homogenizing (i.e., experimental group); and FIG. 9 shows the result of particle size distribution of the fish oil composition without homogenizing (i.e., control group). As shown in FIG. 6 and FIG. 8, the particle size of the experimental group is mainly concentrated at 100 nm to 400 nm, preferably between 180 nm and 250 nm, and the average particle size is 216.5 nm. In comparison, as shown in FIG. 7 and FIG. 9, the particle size of the control group is mainly concentrated at 100 nm to 700 nm, and the average particle size is 382.8 nm. The experimental results show that the high pressure homogenization treatment is the key to reduce the average particle size of the micron fish oil composition of the present invention. The average particle size of the micron fish oil composition can be reduced from 382.8 nm to 216.5 nm, which significantly reduces the fish oil particle size. Therefore, the micron fish oil composition of the present invention has better bioabsorbability and bioavailability.

### Example 4

### Analysis of Human Body Absorption Rate and Utilization Rate of Micron Fish Oil Composition

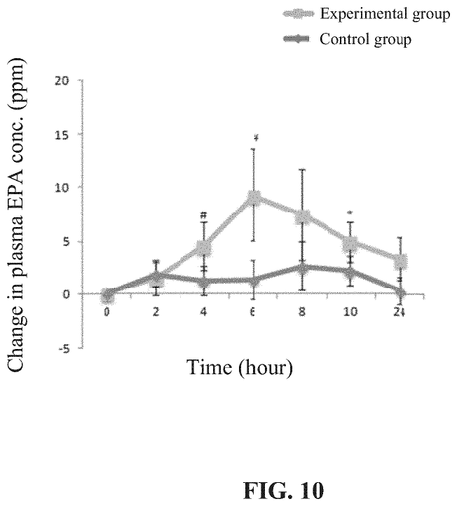

This experiment took the form of a crossover study and a placebo-controlled study. A total of four healthy subjects were recruited to ingest 4.2 g of the micron fish oil composition (i.e., experimental group) homogenized by high pressure (300 bar to 350 bar) of the present invention and fish oil composition without high pressure homogenization treatment (i.e., control group). Next, before the fish oil composition was taken (fasting) and 24 hours after the fish oil composition was taken, the concentration variation of EPA and/or DHA in the plasma and red blood cells of each subject was examined, thereby evaluating the biological absorption rate and utilization rate of the fish oil composition in the experimental group and the control group. Subjects were given a low-fat diet during the trial and avoided eating foods such as fish, flaxseed, spirulina, blackcurrant oil and nuts that would cause experimental errors. The results of this experiment are shown in FIG. 10 to FIG. 15.

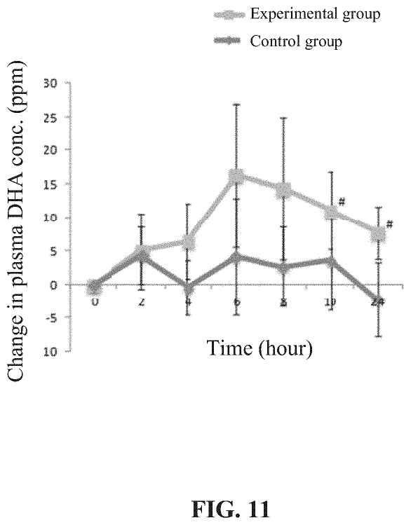

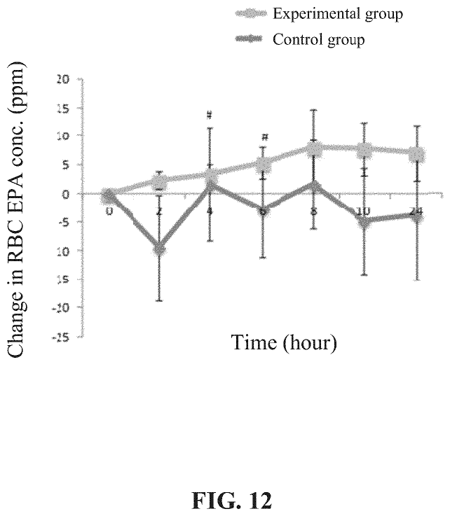

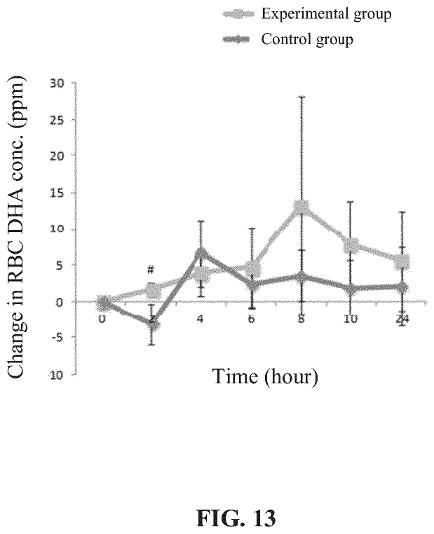

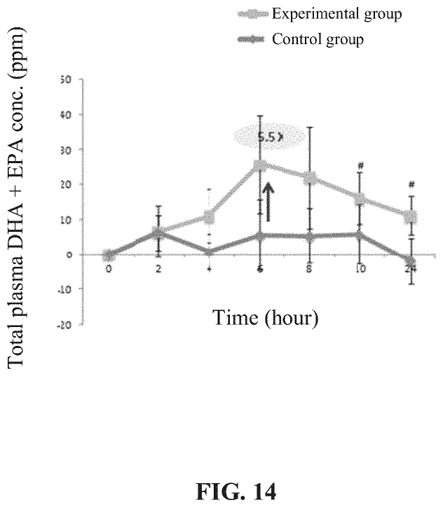

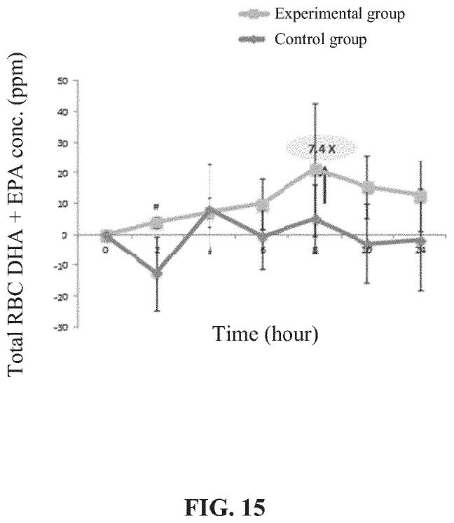

FIG. 10 shows changes in plasma EPA concentration in the subject after administration of the micron fish oil composition (i.e., experimental group) of the present invention by high pressure (300 bar to 350 bar) homogenization or the fish oil composition (i.e., control group) which has not been subjected to high pressure homogenization; FIG. 11 shows changes in plasma DHA concentration in the subject after administration of the micron fish oil composition (i.e., experimental group) of the present invention by high pressure (300 bar to 350 bar) homogenization or the fish oil composition (i.e., control group) which has not been subjected to high pressure homogenization; FIG. 12 shows changes in EPA concentration in red blood cells in the subject after administration of the micron fish oil composition (i.e., experimental group) of the present invention by high pressure (300 bar to 350 bar) homogenization or the fish oil composition (i.e., control group) which has not been subjected to high pressure homogenization; FIG. 13 shows changes in DHA concentration in red blood cells in the subject after administration of the micron fish oil composition (i.e., experimental group) of the present invention by high pressure (300 bar to 350 bar) homogenization or the fish oil composition (i.e., control group) which has not been subjected to high pressure homogenization; FIG. 14 shows changes in plasma EPA and DHA concentration in the subject after administration of the micron fish oil composition (i.e., experimental group) of the present invention by high pressure (300 bar to 350 bar) homogenization or the fish oil composition (i.e., control group) which has not been subjected to high pressure homogenization (i.e., the sum of changes in plasma EPA and DHA concentrations in each hour in FIGS. 10 and 11); and FIG. 15 shows changes in EPA and DHA concentration in red blood cells in the subject after administration of the micron fish oil composition (i.e., experimental group) of the present invention by high pressure (300 bar to 350 bar) homogenization or the fish oil composition (i.e., control group) which has not been subjected to high pressure homogenization (i.e., the sum of changes in EPA and DHA concentrations in each hour of red blood cells in FIGS. 12 and 13).

As shown in FIG. 10, the plasma EPA concentration of the experimental group was higher than that of the control group within 24 hours after taking the fish oil composition; as shown in FIG. 11, the plasma DHA concentration of the experimental group was higher than that of the control group within 24 hours after taking the fish oil composition; as shown in FIG. 12, the EPA concentration in the red blood cells of the experimental group was higher than that of the control group within 24 hours after taking the fish oil composition; as shown in FIG. 13, within 24 hours after taking the fish oil composition, except for the 4th hour, the DHA concentration in the red blood cells of the experimental group was higher than that of the control group; as shown in FIG. 14, within 24 hours after taking the fish oil composition, the concentration of EPA and DHA in the plasma of the experimental group was higher than that of the control group. Especially at the 6th hour, the concentration of EPA and DHA in the plasma of the experimental group was 5.5 times than that of the control group. As shown in FIG. 15, within 24 hours after taking the fish oil composition, the concentration of EPA and DHA in the red blood cells of the experimental group was higher than that of the control group. Especially at the 8th hour, the concentration of EPA and DHA in the red blood cells of the experimental group was 7.4 times than that of the control group. The experimental results of this example show that the micron fish oil composition of the present invention can effectively enhance the bioabsorbability and bioavailability in a subject (e.g., a human body).

Therefore, the foregoing experimental results show that after taking the micron fish oil composition after high pressure homogenization of the present invention, the concentration of EPA and DHA in plasma or red blood cells is higher than that of the fish oil composition before high pressure homogenization in 24 hours. After comparison, it can be found that the micron fish oil composition after high pressure homogenization can increase the EPA and DHA content in plasma by 5.5 times, and increase the EPA and DHA content in red blood cells by 7.4 times, which can confirm that the micron fish oil composition of the present invention can significantly enhance the absorption rate and utilization rate of fish oil for human body.

In summary, the micron fish oil composition of the present invention has been confirmed by experiments to have at least the following effects: stable emulsification condition, excellent dispersion stability, small average particle size, and effective promotion of bioabsorbability and bioavailability in a subject.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for preparing a micron fish oil composition, comprising the following steps:
mixing a composite plant colloid with a fish oil and subjecting to an emulsification treatment to obtain an emulsion; and
homogenizing the emulsion at a pressure of 300 bar to 350 bar to obtain the micron fish oil composition.

2. The method according to claim 1, wherein the composite plant colloid is selected from the group consisting of arabic gum, guar gum, xanthan gum, and any combination thereof.

3. The method according to claim 1, wherein the composite plant colloid is in an amount of 6% to 10% by weight, and the fish oil is in an amount of 3% to 15% by weight.

4. The method according to claim 1, wherein the micron fish oil composition has an average particle size between 180 and 250 nm.

5. The method according to claim 1, wherein the emulsification treatment has a time duration between 25 and 45 minutes.

6. The method according to claim 1, wherein the emulsification treatment is carried out at 15° C. to 30° C.

7. The method according to claim 1, wherein the homogenizing step is carried out at 15° C. to 40° C.

8. A micron fish oil composition prepared by a process comprising the steps of:
mixing a composite plant colloid with a fish oil and subjecting to an emulsification treatment to obtain an emulsion; and
homogenizing the emulsion at a pressure of 300 bar to 350 bar to obtain the micron fish oil composition.

9. The micron fish oil composition according to claim 8, wherein the composite plant colloid is selected from the group consisting of arabic gum, guar gum, xanthan gum, and any combination thereof.

10. The micron fish oil composition according to claim 8, wherein the composite plant colloid is in an amount of 6% to 10% by weight, and the fish oil is in an amount of 3% to 15% by weight.

11. The micron fish oil composition according to claim 8, wherein the micron fish oil composition has an average particle size between 180 and 250 nm.

12. The micron fish oil composition according to claim 8, wherein the emulsification treatment has a time duration between 25 and 45 minutes.

13. The micron fish oil composition according to claim 8, wherein the emulsification treatment is carried out at 15° C. to 30° C.

14. The micron fish oil composition according to claim 8, wherein the homogenizing step is carried out at 15° C. to 40° C.

15. A method for enhancing a bioabsorbability and a bioavailability in a subject, comprising administering to the subject in need thereof a food product comprising an effective amount of the micron fish oil composition according to claim 8.
